(19) United States
(12) Patent Application Publication
(10) Pub. No.: US 2016/0083683 A1
(43) Pub. Date: Mar. 24, 2016

(54) Anaerobic Digester System

(71) Applicants: Matthew Augustine, Rochester, NY (US); Rodrigo Labatut, Rochester, NY (US); Sara Silverstone, Rochester, NY (US); Robert Wright, Rochester, NY (US)

(72) Inventors: Matthew Augustine, Rochester, NY (US); Rodrigo Labatut, Rochester, NY (US); Sara Silverstone, Rochester, NY (US); Robert Wright, Rochester, NY (US)

(21) Appl. No.: 14/862,924

(22) Filed: Sep. 23, 2015

Related U.S. Application Data
(60) Provisional application No. 62/054,100, filed on Sep. 23, 2014.

(51) Int. Cl. C12M 1/00; C12M 1/36

(52) U.S. Cl. CPC C12M 21/12; C12M 41/48

(57) ABSTRACT

Embodiments provide a bioreactor system for the bioconversion of biomass into products and renewable energy, and a method for the same.

### CROSS-REFERENCE TO RELATED APPLICATIONS

This application claims priority to Provisional Patent Application No. 62/054,100, filed Sep. 23, 2014, which is hereby incorporated by reference in its entirety.

### FIELD OF THE INVENTION

The present disclosure relates to an anaerobic digester system, more specifically a portable system for demonstrating to students how biomass is converted into products and renewable energy by microorganisms maintained in an oxygen-free environment.

### BACKGROUND OF THE INVENTION

Renewable energy is defined as energy that comes from resources which are naturally replenished on a human timescale. The five major categories are solar, wind, water, geothermal, and biomass. Through collaborations with university researchers in renewable energy technology development and industrial practitioners, these technologies are able to be scaled to create effective and innovative teaching tools.

A two-stage (vessel) anaerobic digester (AD) is typically more stable than a single-stage design. During the anaerobic digestion process, methane-producing microorganisms (i.e., methanogens) consume the acids produced by the hydrolytic and fermenting bacteria to produce methane gas. In a single-vessel design, these microbial groups need to be at equilibrium to prevent acids to accumulate and decrease the system's pH. Metabolism of methanogens is optimized in a narrow pH range, i.e., between 6.5 and 7.6. Furthermore, their metabolism will slow down considerably at pH lower than 6.0. Contrarily, acid-producing bacteria have their optimal near pH 6.0. If a single-vessel AD is overloaded (fed with too much organic material), acids will accumulate, and pH will decrease to a point in which the methanogenic population will collapse, causing methane/biogas production to stop. This is called a crash and can take weeks to recover.

A similar scenario can also occur if a feed stock is introduced with high protein content, causing excessive formation of ammonia that leads to an environment that is too alkaline. A two vessel AD design separates the hydrolysis and fermentation stages from the methanogenesis stage and mitigates large changes in pH within the methanogenesis environment. A need exists for a more stable anaerobic digester that mitigates large changes in pH.

A further need exists for a system that is able to supply students with enhanced STEM (Science Technology Engineering Mathematics) skills as well as helping the students employ the new economic model of the “triple bottom line” of sustainable business practices through developed and accumulated comprehensive energy related knowledge as well as through products and services for energy education, production, saving techniques, methods, and processes.

### BRIEF SUMMARY OF THE INVENTION

The disclosure may provide a demonstration system for the anaerobic bioconversion process, where biomass is broken down to generate renewable energy. The system may comprise a physical bench-scale anaerobic digester, a simulated/game anaerobic digester, and related anaerobic digestion kits with experiments and lesson plans aligned with CCSM (Common Core State Mathematics) and NGSS (Next Generation Science Standards).

The anaerobic digester (AD) may convert biodegradable organic material into biogas, a combustible gas and a renewable fuel consisting of approximately 50% to 60% methane with the balance being mostly carbon dioxide and small amounts of other gases. In embodiments, the AD-250 may comprise two vessels, a hydrolysis and a methanogenesis vessel. The environments in these vessels may be controlled and designed to harbor differing microbial populations to perform specific functions.

A two vessel AD is typically more stable than a single vessel design in which the entire microbial population has to exist in equilibrium. A two vessel AD design may separate the hydrolysis/fermentation stages from the methanogenesis stage and mitigates large changes in pH within the methanogenesis environment. In embodiments, the AD-250 may be a bench-scale anaerobic digester prototype that may be configured either as a single-stage digester or as a two-stage digester, wherein digestion is divided into hydrolysis/fermentation and methanogenesis stages, similar to state-of-the-art commercial facilities.

In embodiments, the AD-250 may be equipped with an external transparent ˜0.5 liter gas accumulator 4. In embodiments, the hydrolysis/fermentation vessel (hereafter called hydrolysis vessel) may have a capacity of 9 liters for decomposition of the organic material. In embodiments, the methanogenesis vessel may have a capacity of 6 liters and may contain a heater, a fluid sampling port, a biofilm polypropylene structured media, and a motor-driven agitator/distributor. The biogas may pass through the condenser/scrubber column prior to measurement and collection, removing condensation and sulfur related gases. The system may further include a fuel cell and burner device capable of converting and quantifying the energy produced from the biogas flare into electricity.

The disclosure may further provide a computer program product relative to a proprietary quick start media and graphical user interface (specifically LabVIEW software-based) for steps relative to the biomass conversion, including the inoculation of the methanogenesis vessel. The computer program product may also include teaching materials. The graphical user interface (GUI) may provide a user with flexibility in experiment design and control.

The disclosure may further provide a method for breaking down biomass to generate renewable energy using the demonstration anaerobic digester system.

In embodiments, the disclosure may provide a simplistic method, digestion system, and computer program product for breaking down biomass to generate renewable energy.

These and other aspects of the disclosed subject matter, as well as additional novel features, will be apparent from the description provided herein. The intent of this summary is not to be a comprehensive description of the subject matter, but rather to provide a short overview of some of the subject matter's functionality. Other systems, methods, features and advantages here provided will become apparent to one with skill in the art upon examination of the accompanying FIGURES and detailed description. It is intended that all such additional systems, methods, features and advantages that are included within this description, be within the scope of any claims filed now or later.

### BRIEF DESCRIPTION OF THE DRAWINGS

The novel features believed characteristic of the disclosed subject matter will be set forth in any claims that are filed now or later. The disclosed subject matter itself, however, as well as a preferred mode of use, further objectives, and advantages thereof, will best be understood by reference to the following detailed description of an illustrative embodiment when read in conjunction with the accompanying drawings, wherein:

FIG. 1 depicts an embodiment of a bioreactor system in accordance with embodiments.

FIG. 2 depicts a gas flow diagram of the anaerobic digester system in accordance with embodiments.

FIG. 3A depicts a rendering of a burner/fuel cell assembly of an anaerobic digester system in accordance with embodiments.

FIG. 3B depicts an internal view of a burner/fuel cell assembly in accordance with embodiments.

FIG. 4A depicts a front view of an exemplary hardware layout of the anaerobic digester system in accordance with embodiments.

FIG. 4B depicts a perspective view of an exemplary hardware layout of the anaerobic digester system in accordance with embodiments.

FIG. 4C depicts a top view of an exemplary hardware layout of the anaerobic digester system in accordance with embodiments.

FIG. 5 depicts a screen shot displayed on the monitor of the computing system showing an overview panel in accordance with embodiments.

FIG. 6 depicts a screen shot displayed on the monitor of the computing system showing a calibration panel in accordance with embodiments.

FIG. 7 depicts a screen shot displayed on the monitor of the computing system showing a pH calibration panel in accordance with embodiments.

FIG. 8 depicts a screen shot displayed on the monitor of the computing system showing a setup panel in accordance with embodiments.

### DETAILED DESCRIPTION OF ILLUSTRATIVE EMBODIMENTS

Reference now should be made to the drawings, in which the same reference numbers are used throughout the different figures to designate the same components.

It will be understood that, although the terms first, second, third, etc. may be used herein to describe various elements, these elements should not be limited by these terms. These terms are only used to distinguish one element from another element. Thus, a first element discussed below could be termed a second element without departing from the teachings of the present disclosure.

The terminology used herein is for the purpose of describing particular embodiments only and is not intended to be limiting. As used herein, the singular forms “a”, “an”, and “the” are intended to include the plural forms as well, unless the context clearly indicates otherwise. It will be further understood that the terms “comprises” and/or “comprising” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Although described with reference to personal computers and the Internet, one skilled in the art could apply the principles discussed herein to any computing or mobile computing environment. Further, one skilled in the art could apply the principles discussed herein to communication mediums beyond the Internet.

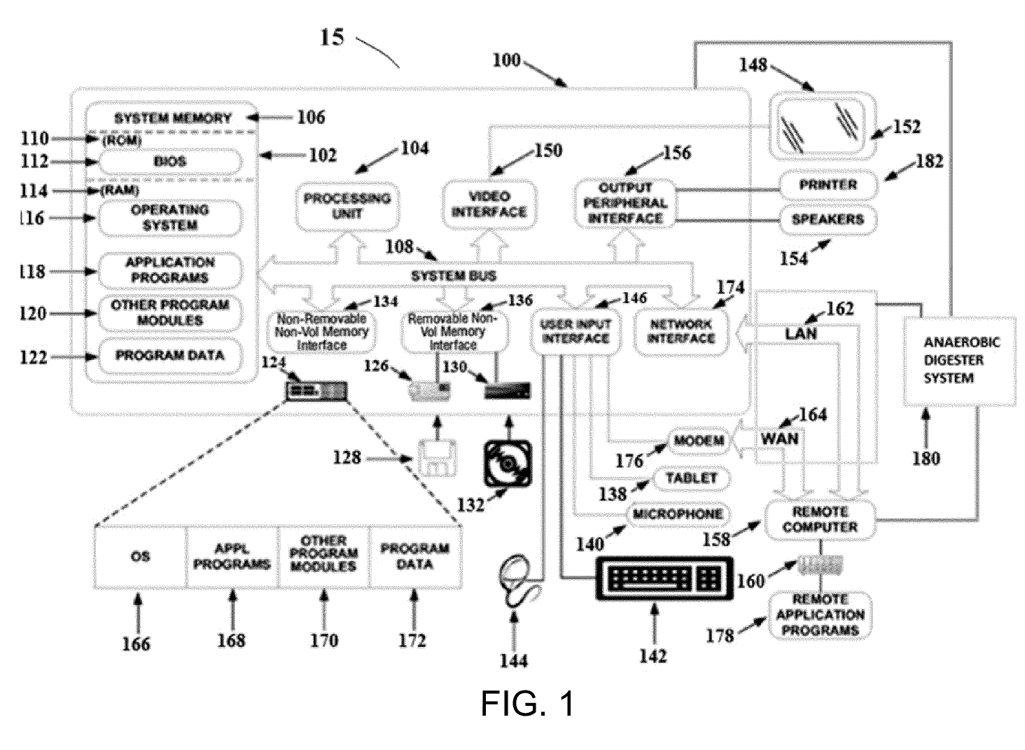

FIG. 1 depicts an embodiment of a bioreactor system 15 within a computing environment for implementing the disclosure and bioreactor system 15 which may include a general purpose computing device in the form of a computing system 100, commercially available from Intel, IBM, AMD, Motorola, Cyrix, etc. An exemplary bioreactor system 10 may include a computing system 100 in communication with a digester/bioreactor 180 as described herein below. Components of the computing system 102 may include, but are not limited to, a processing unit 104, a system memory 106, and a system bus 108 that couples various system components including the system memory 106 to the processing unit 104. The system bus 108 may be any of several types of bus structures including a memory bus or memory controller, a peripheral bus, or a local bus using any of a variety of bus architectures.

Computing system 100 typically includes a variety of computer readable media. Computer readable media can be any available media that can be accessed by the computing system 100 and includes both volatile and nonvolatile media, and removable and non-removable media. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data.

Computer memory includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by the computing system 100.

The system memory 106 includes computer storage media in the form of volatile and/or nonvolatile memory such as read only memory (ROM) 110 and random access memory (RAM) 114. A basic input/output system 112 (BIOS), containing the basic routines that help to transfer information between elements within computing system 100, such as during start-up, is typically stored in ROM 110. RAM 114 typically contains data and/or program modules that are immediately accessible to and/or presently being operated on by processing unit 104. By way of example, and not limitation, an operating system 116, application programs 118, other program modules 120 and program data 122 are shown.

Computing system 100 may also include other removable/non-removable, volatile/nonvolatile computer storage media. By way of example only, a hard disk drive 124 that reads from or writes to non-removable, nonvolatile magnetic media, a magnetic disk drive 126 that reads from or writes to a removable, nonvolatile magnetic disk 128, and an optical disk drive 130 that reads from or writes to a removable, nonvolatile optical disk 132 such as a CD ROM or other optical media could be employed to store the invention of the present embodiment. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 124 is typically connected to the system bus 108 through a non-removable memory interface such as interface 134, and magnetic disk drive 126 and optical disk drive 130 are typically connected to the system bus 108 by a removable memory interface, such as interface 136.

The drives and their associated computer storage media, discussed above, provide storage of computer readable instructions, data structures, program modules and other data for the computing system 100. For example, hard disk drive 124 is illustrated as storing operating system 166, application programs 168, other program modules 170, and program data 172. Note that these components can either be the same as or different from operating system 116, application programs 118, other program modules 120, and program data 122. Operating system 166, application programs 168, other program modules 170, and program data 172 are given different numbers here to illustrate that, at a minimum, they are different copies.

A user may enter commands and information into the computing system 100 through input devices such as a tablet, or electronic digitizer, 138, a microphone 140, a keyboard 142, and pointing device 144, commonly referred to as a mouse, trackball, or touch pad. These and other input devices are often connected to the processing unit 104 through a user input interface 146 that is coupled to the system bus 108, but may be connected by other interface and bus structures, such as a parallel port, game port or a universal serial bus (USB).

A monitor 148 or other type of display device is also connected to the system bus 108 via an interface, such as a video interface 150. The monitor 148 may also be integrated with a touch-screen panel or the like. Note that the monitor and/or touch screen panel can be physically coupled to a housing in which the computing system 100 is incorporated, such as in a tablet-type personal computer. In addition, computers such as the computing system 100 may also include other peripheral output devices such as speakers 154 and printer 182, which may be connected through an output peripheral interface 156 or the like.

Computing system 100 may operate in a networked environment using logical connections to one or more remote computers, such as a remote computing system 158. The remote computing system 158 may be a personal computer (including, but not limited to, mobile electronic devices), a server, a router, a network PC, a peer device or other common network node, and typically includes many or all of the elements described above relative to the computing system 100, although only a memory storage device 160 has been illustrated. The logical connections depicted include a local area network (LAN) 162 connecting through network interface 174 and a wide area network (WAN) 164 connecting via modem 176, but may also include other networks. Such networking environments are commonplace in offices, enterprise-wide computer networks, intranets and the Internet.

For example, in the present embodiment, the computer system 100 may comprise the source machine from which data is being generated/transmitted and the remote computing system 158 may comprise the destination machine. Note however that source and destination machines need not be connected by a network or any other means, but instead, data may be transferred via any media capable of being written by the source platform and read by the destination platform or platforms.

In another example, in the present embodiment, the remote computing system 158 may comprise the source machine from which data is being generated/transmitted and the computer system 100 may comprise the destination machine.

In a further embodiment, in the present disclosure, the computing system 100 may comprise both a source machine from which data is being generated/transmitted and a destination machine and the remote computing system 158 may also comprise both a source machine from which data is being generated/transmitted and a destination machine.

For the purposes of this disclosure, it is appreciated that the terms “device”, “processor based mobile device”, “mobile device”, “electronic device”, “processor based mobile electronic device”, “mobile electronic device”, “location-capable wireless device”, “user device”, and “user electronic device” may be synonymous with remote computer 158.

The central processor operating pursuant to operating system software such as IBM OS/2®, Linux®, UNIX®, Microsoft Windows®, Apple Mac OSX® and other commercially available operating systems provides functionality for the services provided by the present disclosure. The operating system or systems may reside at a central location or distributed locations (i.e., mirrored or standalone).

Software programs or modules instruct the operating systems to perform tasks such as, but not limited to, facilitating client requests, system maintenance, security, data storage, data backup, data mining, document/report generation and algorithms. The provided functionality may be embodied directly in hardware, in a software module executed by a processor or in any combination of the two.

Furthermore, software operations may be executed, in part or wholly, by one or more servers or a client's system, via hardware, software module or any combination of the two. A software module (program or executable) may reside in RAM memory, flash memory, ROM memory, EPROM memory, EEPROM memory, registers, hard disk, a removable disk, a CD-ROM, DVD, optical disk or any other form of storage medium known in the art. An exemplary storage medium is coupled to the processor such that the processor can read information from, and write information to, the storage medium. In the alternative, the storage medium may be integral to the processor. The processor and the storage medium may also reside in an application specific integrated circuit (ASIC). The bus may be an optical or conventional bus operating pursuant to various protocols that are well known in the art.

Referring to FIG. 1, bioreactor system 15 may include the computing system 100 in operable communication with the two-stage anaerobic digester/bioreactor 180. The two-stage anaerobic digester/bioreactor 180 may comprise separate hydrolysis and methanogenesis vessels 3,2 with the two vessels 3,2 being able to transfer matter via a peristaltic pump 8. In embodiments, organic waste may be put into the hydrolysis vessel 3, wherein the organic waste decomposes and is subsequently transferred to the methanogenesis vessel 2.

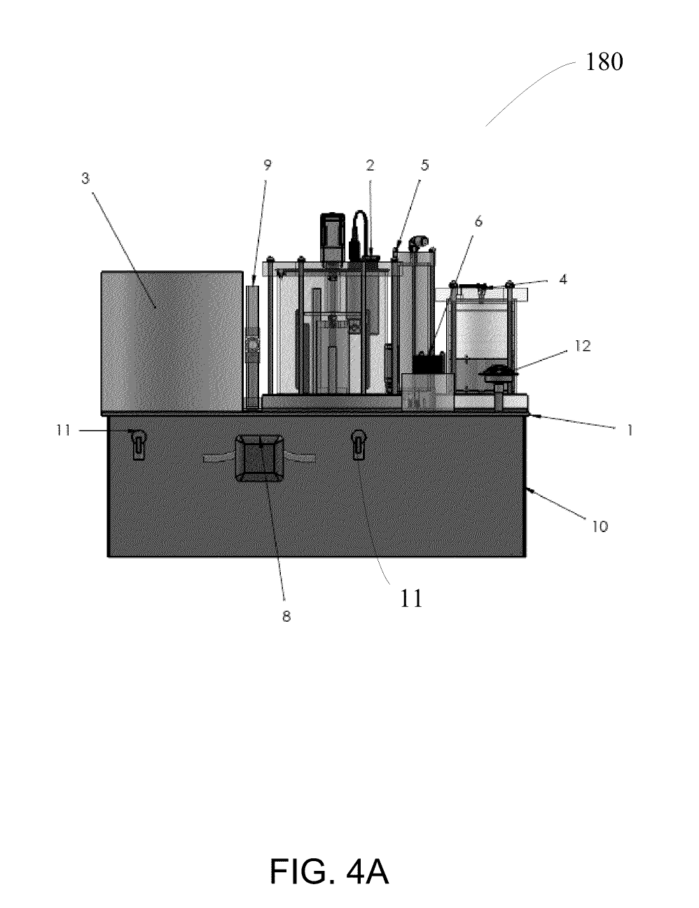

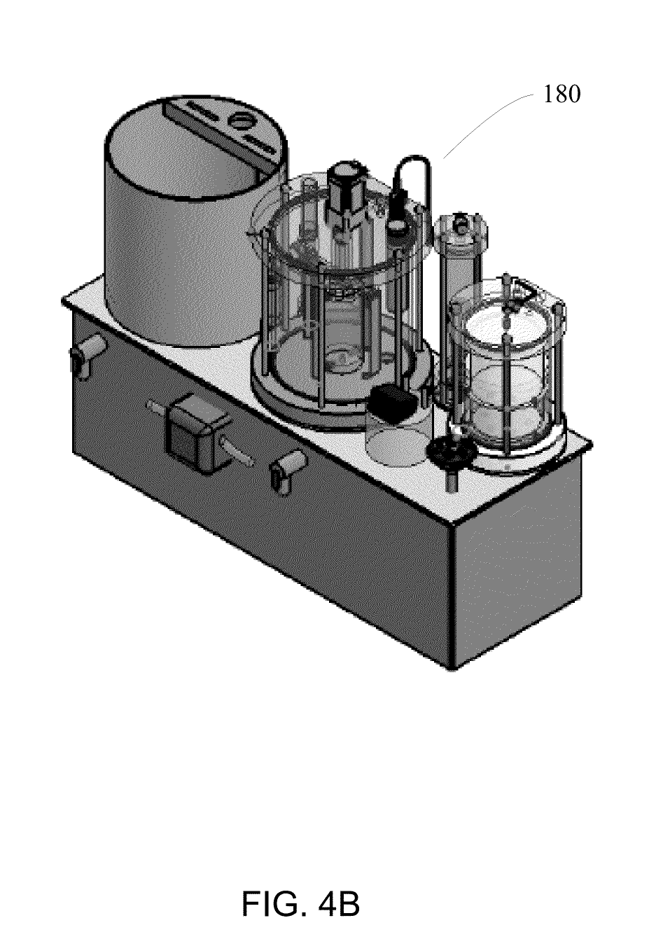

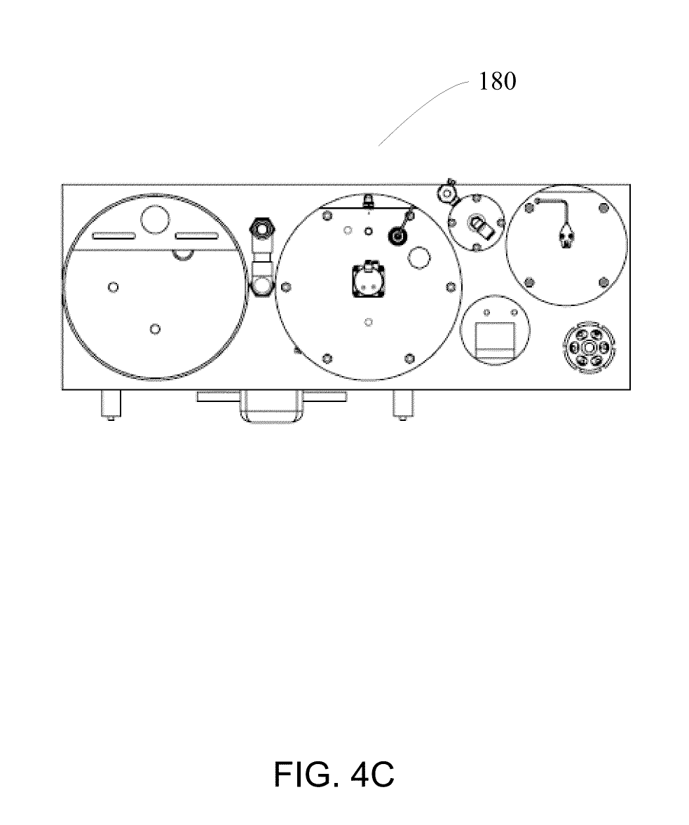

In embodiments, bioreactor system 15 may include the hydrolysis vessel (HV) 3 (see FIG. 4) may be fitted with a removable cover to mitigate volatile material loss and permit either the addition or removal of material. Fluid and material contained within the hydrolysis vessel 3 may flow into the pump tubing through a stand pipe within the hydrolysis vessel 3. In embodiments, the pump 8 may automatically transfer preprogrammed fluid volume from the HV 3 to the MV (methanogenesis vessel) 2 based on the user's determined HRT (hydraulic retention time). A given volume of material transferred from the hydrolysis vessel 3 may cause the same volume of fluid to be discharged from the methanogenesis vessel 2. The displaced fluid in the methanogenesis vessel 2 may then flow out through the effluent pipe to drain. In embodiments, the HV 3 may also be fitted with a probe to monitor pH. Sample digester fluid may be dispensed from the front panel.

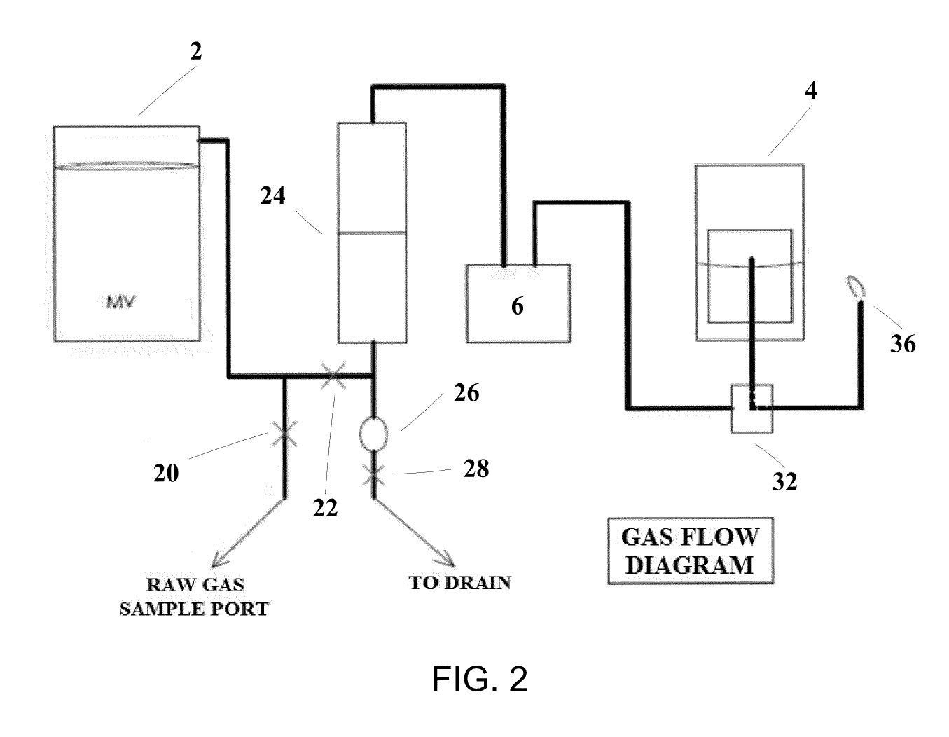

The digester fluid may be transferred from the hydrolysis vessel 3 to the methanogenesis vessel 2 (see FIG. 2). Once in the methanogenesis vessel 2, methanogens may consume the organic matter (under anaerobic conditions) in order to multiply. The consumption of the organic matter may then produce biogas. In embodiments, the methanogenesis vessel 2 may be an enclosed unit and may include elements such as, but not limited to, a mixer, a heater, a temperature sensor, a fluid sampler port, a fluid overflow pipe, a top fluid access port, and a biofilm polypropylene (PP) structured media reef. The random packing PP support media may have a surface area of ˜1.2 square feet of material. In embodiments, the MV 2 may also be fitted with a probe to monitor pH. The vessel may be wrapped with an insulator, such as a Kydex insulator, to maintain the temperature of the feedstock in the vessel and avert exposure to light. The heater may be controlled by a software PID with onscreen controls.

In embodiments, the methanogenesis vessel 2 may include a mixer. Mixing may be variable from 0 to 15 RPM. Sample digester fluid may be dispensed from the front panel and raw biogas may be dispensed from the headspace via syringe access. The pump 8 may automatically transfer preprogrammed fluid volume from HV 3 to MV 2 based on the user's determined HRT (hydraulic retention time). As material is transferred from the hydrolysis vessel 3, it may cause the same volume of fluid to be discharged from the methanogenesis vessel 2. The displaced fluid in the methanogenesis vessel 2 may flow out through the effluent pipe to drain. In order to allow for the ideal growth of mesophiles, the mesophiles may be exposed to moderate temperatures, typically between 20 degrees and 45 degrees Celsius. This may produce ideal conditions for the consumption of organic material.

Material added to the TS-AD250 may be introduced in either a whole, chopped or macerated state (feedstock). Certain feedstock may contain some material that may digest slowly; for example apple stalks and seeds may take months to break down. Soil and grit may be indigestible and may be best removed from any feedstock. The accidental introduction of these materials may not be harmful. However, their introduction may ultimately necessitate their removal from the hydrolysis vessel 3. Feedstock packing materials may be removed and not introduced; packing materials may block plumbing.

Freshwater may be added to the hydrolysis vessel 3 to replace the hydrolyzed material transferred to the methanogenesis vessel 2. The volume of water added dictates the HRT or hydraulic retention time in both the methanogenesis vessel 2 and the hydrolysis vessel 3. The hydraulic retention time may usually be expressed in days. A formula to calculate the HRT is as follows:

HRT (days)=[Volume of Vessel (L)]/[Water (or biomass) flow rate (L/day)]

So for example, if 150 ml of water are added daily to the methanogenesis vessel 2, its HRT will be 40 days (6 L/0.15 L/day=40 days). For the hydrolysis vessel 3, the HRT would be 60 days (9 L/0.15 L/day=60 days). This formula may apply whether it is water or biomass that is being fed to the anaerobic digester system 180. The HRT of both vessels may double if half of the volume of water is added daily, or may be reduced to half if the daily volume is doubled.

In embodiments, the anaerobic digester system 180 may be utilized as a single-stage digester.

In embodiments, the temperature controller of the system 180 may not be set higher than 55 degrees Celsius. The liquid in the methanogenesis vessel 2 may be the primary thermal path by which the heater's energy is dissipated and then detected by the temperature probe. The absence of liquid may remove the primary path and may cause the heater temperature to rise to a level that may damage the vessel and may cause it to leak.

FIG. 2 depicts a gas flow diagram of the anaerobic digester system 180 in accordance with embodiments. Once organic material has decomposed in the hydrolysis vessel 3 (not shown), the digester fluid may be sent through a connection tube to the methanogenesis vessel (MV) 2. The methanogenesis vessel 2 may be used to house microbes that form methane from the decomposed material and may be able to store up to 6 liters of digester fluid. A sample control valve 20 may be attached to the line extending from the methanogenesis vessel 2. The sample control valve 20 may be used to take a sample of the digester fluid at any point in time during the degradation process. Alternatively, a sample of raw biogas may be sampled from the headspace of the methanogenesis vessel 2 via top port syringe access.

The temperature of the methanogenesis vessel 2 may be monitored continuously or periodically, and for example may be checked daily. Temperatures within the vessel may be critical for optimal function of mesophilic microbes, which may occur between the temperatures of 32 degrees to 43 degrees Celsius. In embodiments, a maximum conversion may occur at approximately 35 degrees Celsius. For convenience, a temperature sensor may exist in the methanogenesis vessel 2 that may send temperature data to the computing system 100. The data may be viewed on the monitor 148 of the computing system 100. Temperature of the methanogenesis vessel 2 may be controlled by use of the temperature data and a suitable temperature control apparatus.

In embodiments, a MV control valve 22 may also be attached to a line extending from the methanogenesis vessel 2 and may be used to control the flow of the biogas to other parts of the system 180, such as, but not limited to the condenser/scrubber 24. The condenser/scrubber 24 may be utilized to remove condensation and sulfur related gases from the biogas. Condensation in the biogas may be collected in the condensate bowl 26, wherein the condensate bowl may be emptied by engaging the manual drain valve 28 located on the same line as the condensate bowl 26. When the manual drain valve 28 is open, condensate may empty out of the condensate bowl and into a drain.

Once passed through the condenser/scrubber 24, the biogas may be directed to the milligas counter (MGC) 6 via another line connecting the condenser/scrubber 24 and the milligas counter 6. The milligas counter 6 may accurately record the volume of gas produced from the methanogenesis vessel 2 in milliliters. In embodiments, the information recorded may be streamed directly to the computing system 100 for analysis. The gas may then be sent, via a line and three-way valve 32, to the gas accumulator 4. The gas accumulator 4 may hold up to 500 milliliters of biogas and may be automatically emptied when full by way of sensors that may trigger the gas accumulator 4 to release the biogas. Alternatively, the biogas may be released manually by a user engaging a toggle icon on the screen of the computing system 100 and opening the three way valve 32 toward a line connected to the flare port/condition gas sample port 36. Once the gas reaches this point, the gas may be combusted using the flare port 36 or captured for analysis.

The anaerobic digester system 180 may be used in conjunction with renewable energy education kits, which may include ingredients necessary to run experiments/lessons with the anaerobic digester system 180. In embodiments, the kit may comprise 24 anaerobic reaction vessels, 100 grams of BioDrill Innoculum Powder, 200 grams of BioDrill resuspension powder, 50 grams of cellulose powder, one 100 milliliter bottle of 60 molar acetic acid solution, six 1 milliliter sampling syringes, six 20 milliliter sampling syringes, twelve sterile hypodermic needles, one strainer, six funnels, 48 safety release aluminum crimp-caps, 48 PTFE butyl rubber septa, one hand crimper, one hand de-capper, and one anaerobic digestion and biogas production lecture and lab manual CD. Depending on what experiment is to be performed, the materials for certain experiments may vary.

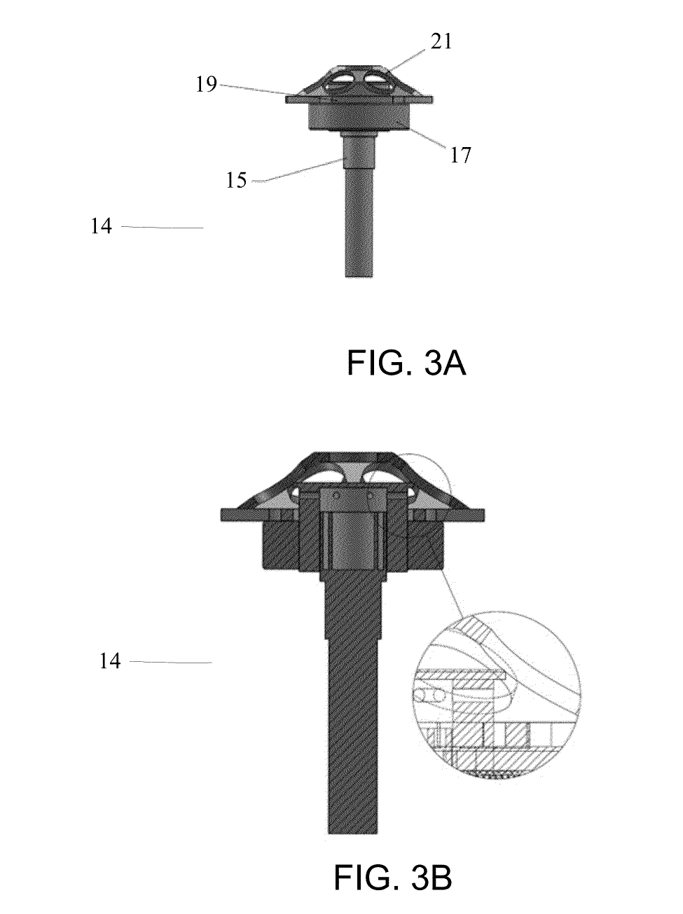

FIG. 3A depicts a detailed rendering of a burner/fuel cell assembly 14 of an anaerobic digester system 180 in accordance with embodiments. The burner/fuel cell assembly 14 may be attached to the end of a line attached to the gas accumulator 4 of the anaerobic digester 180. The burner of the burner/fuel cell assembly 14 may comprise a Bunsen burner 15, a clamp 17, a spacer 19, and a fuel cell holder 21. Clamp 17 may be attached around the circumference of the Bunsen burner to provide stabilization of the fuel cell holder 21. FIG. 3B depicts an internal view of a burner/fuel cell assembly 14 in accordance with embodiments. The fuel cell holder 21 may be circular in shape and may house up to six fuel cells at the same time. In embodiments, the fuel cell holder 21 may house more than six fuel cells at the same time. In embodiments, the interior of the burner/fuel cell assembly 14 may be at least partially covered by the fuel cell holder 21 (FIG. 3B). In embodiments, the burner/fuel cell assembly 14 may comprise flare port 36.

FIGS. 4A, 4B, and 4C depict front, perspective, and top views of an exemplary hardware layout of the anaerobic digester system 180 in accordance with embodiments. In embodiments, for example, the system 180 may comprise a plurality, such as twelve, of mechanical parts. The frame top plate 1 may be a table section of the system 180 and may support much of the mechanical parts of the anaerobic digester system 180. In embodiments, the dimensions of the frame top plate 1 may be 11 inches by 30.5 inches. In embodiments, the anaerobic digester system 180 may comprise small dimensions, making it portable and making it easy to fit in almost any classroom or laboratory. In embodiments, the methanogenesis vessel 2 may be one of the larger vessels found within the system 180 and may comprise a heater, a fluid sampling port, biofilm polypropylene structured media, and a brushless electric motor driven agitator/distributor. In embodiments, the methanogenesis vessel 2 may have a capacity of 6 liters. In embodiments, the hydrolysis vessel 3 may be the largest capsule found within the system 180 and may be used as a housing chamber for the decomposition process of the organic material. In embodiments, the hydrolysis vessel 3 may have a capacity of 9 liters. In embodiments, the gas accumulator 4 may be the third largest capsule found within the system 180 and may be used to house the biogas product that has undergone processing in the methanogenesis vessel 2 and other components of the system 180. In embodiments, the condenser/scrubber 5 may be used to remove condensation and sulfur related gases from the biogas once it has undergone processing in the methanogenesis vessel 2. In between the gas being transferred from the condenser/scrubber 5 to the gas accumulator 4, the gas may be run through the milligas counter 6, which may be connected to the computing system 100 and may be used to record the amount of gas produced by the anaerobic digester system 180.

In embodiments, the WM313 or transfer pump assembly 8 may be the pump utilized to pump biogas from the hydrolysis vessel 3 to the methanogenesis vessel 2. An overflow assembly 9 may be found on the frame top plate 1 and may be used to drain fluid from the methanogenesis vessel 2. The panel assembly 10 may house much of the lines connecting various components within the anaerobic digester system 180. A sample valve 11 may be found below the hydrolysis vessel 3 and may be used to sample hydrolyzed material found within the hydrolysis vessel 3 at any time. A second sample valve 11 may be found below the methanogenesis vessel 2 to allow sampling of the contents. The Bunsen burner/fuel cell assembly 12 may be connected to a line from the gas accumulator 4 and may be used to burn off the biogas.

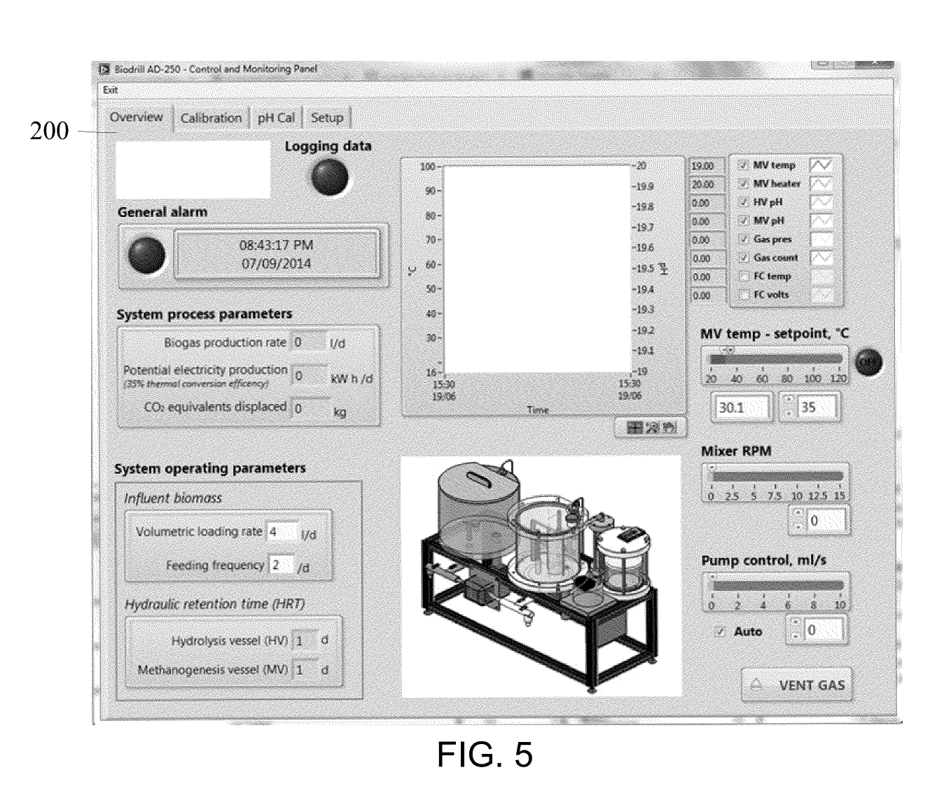

FIG. 5 depicts a screen shot displayed on the monitor 148 of the computing system 100 showing an overview panel 200 in accordance with one embodiment. The overview panel 200 may be associated with the program. The overview panel may display general information pertinent to the anaerobic digester system 180. Subsections found under the overview panel may include, but is not limited to, a logging data section, a general alarm section, a system process parameters section, a system operating parameters section, an MV 2 temperature section, a mixer RPM section, and a pump control section. Sensors throughout the anaerobic digester system 180 may analyze and log specific data at any point in time of processing. The sensors may contribute information to many of the sections found under the overview panel 200. The overview panel 200 may also include a vent gas button engageable by a user. When engaged, this button may release excess gas from the gas accumulator 4 for sample or flare.

The logging data section may include a parameter selection list, wherein a user of the program may engage the specific parameters that the user would like the system 180 to measure such as, but not limited to, methanogenesis vessel 2 temperature, methanogenesis vessel 2 heater, hydrolysis vessel 3 pH, methanogenesis vessel 2 pH, gas pressure, gas count, fuel cell temperature, and volts. The measured parameters may then be displayed in a graph that may chart the parameters versus time.

The general alarm section may include a receiving sensor light that may be used to make a user aware that there is a problem within the system 180. The sensor light may change to an alternative color (such as red) when a problem is detected. The general alarm section may further include a clock and date section that may display the time and date.

The system process parameters section may display certain production parameters pertinent to the system 180. Parameters such as biogas production rate, potential electricity production, and CO₂ equivalents displaced may be monitored by sensors found on the system 180. The biogas production rate may be expressed in liters per day. The potential electricity production may be expressed as 35 percent of the thermal conversion efficiency and may be measured as kilowatt hours per day. The CO₂ equivalents displaced may be expressed in kilograms.

The system operating parameters section may display parameters of influent biomass as well as calculated parameters for hydraulic retention time (HRT). The parameters that may be displayed for the influent biomass may include volumetric loading rate and feeding frequency, which may be entered manually by a user. Using these parameters, the system 100 may calculate the hydraulic retention time (HRT) for the hydrolysis vessel 3 and the methanogenesis vessel 2.

The MV 2 temperature section may allow a user to set the methanogenesis vessel 2 to a set temperature. Sensors in the system 180 may monitor the temperature of the fluid inside of the methanogenesis vessel 2 in response to the set temperature of the methanogenesis vessel 2.

The mixer RPM section may allow a user to manually adjust the speed of the mixer found in the methanogenesis vessel 2 of the system 180. A user may set the rotations per minute (RPM) of the mixer from anywhere between 0 and 15 RPM. Different RPMs may be used at different times during the processing stage of the material found in the methanogenesis vessel 2.

The pump control section may allow a user to manipulate the speed of the pump 8 found between the hydrolysis vessel 3 and the methanogenesis vessel 2. The speed of the pump 8 may be set from anywhere between 0 and 10 milliliters per second. The user may choose to allow the system to automatically change the speed of the pump 8 based on the conditions within the system or may choose to set the pump control manually.

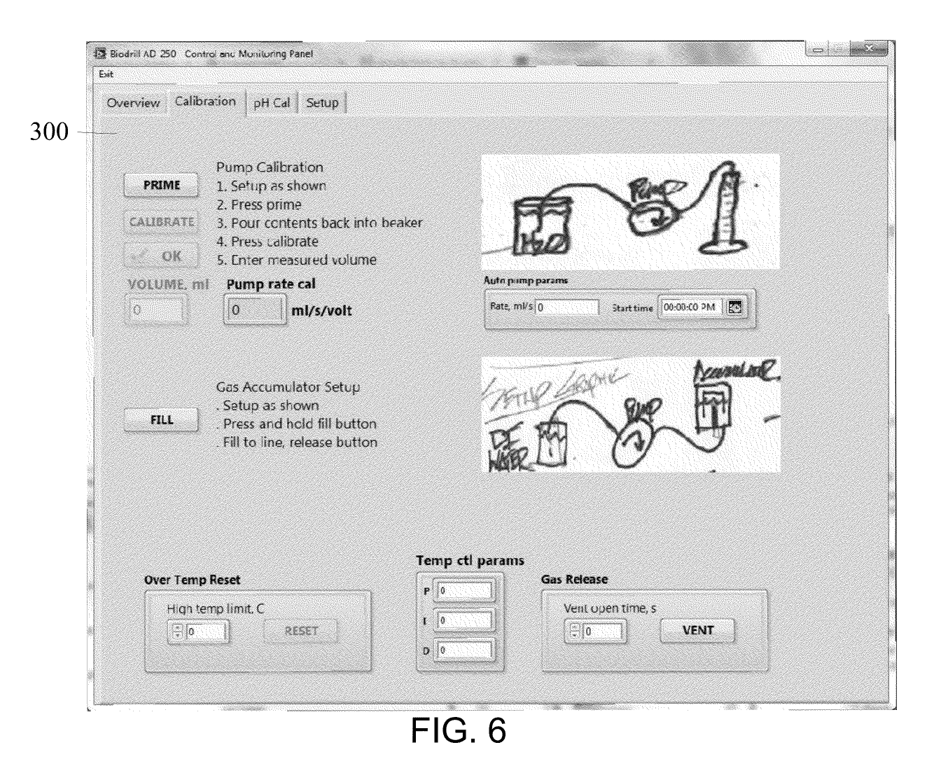

FIG. 6 depicts a screen shot displayed on the monitor 148 of the computing system showing a calibration panel 300 in accordance with embodiments. The calibration tab found within the calibration panel 300 may display instructions to a user that may help a user to calibrate the transfer pump 8 of the system 180. In one section of the calibration tab, the transfer pump 8 may be calibrated, according to the calibration tab, using a container of water and a beaker. On-screen instructions may aid a user in the process. According to the calibration tab, a user may be told to: 1. Reproduce the setup as shown in the window adjacent the instructions; 2. Press an engageable prime button; 3. Pour contents back into beaker; 4. Press calibrate; and 5. Enter measured volume.

Once these measures are taken, the pump may be calibrated and the pump rate may be displayed as a result of the calibration. A second process necessary to start a “clean run” of the anaerobic digester system 180 may be the filling of the gas accumulator 4. According to the calibration tab, a user may be told to reproduce the setup as shown in the window adjacent the instructions, press and hold the fill button, and fill the gas accumulator 4 to the fill line with DI water (and at that point, releasing the button).

The calibration tab may include three other sections: an over temperature reset, a temperature control parameters, and a gas release. The over temperature reset may allow a user of the system 180 to set a limit on the temperature of the methanogenesis vessel 2. A user may also reset the temperature by engaging the “reset” icon.

The calibration tab may further comprise a temperature control parameters section that may allow a user to manually control the heating element of the system 180. The P, I, and D may each be separately controlled. A gas release section may also be accessible within the calibration tab. A user may input the amount of time, in seconds, that the vent may be open in order to release gas. Once the user engages the vent icon, the vent may open and release the gas.

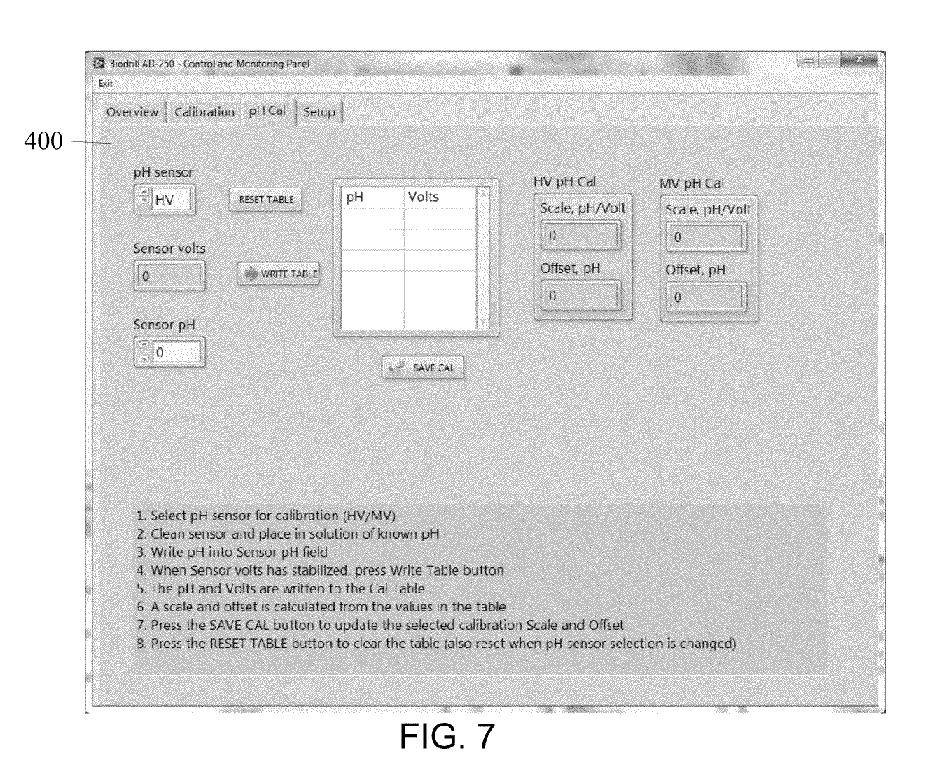

FIG. 7 depicts a screen shot displayed on the monitor 148 of the computing system showing a pH calibration panel 400 in accordance with embodiments. The user may first choose whether to calibrate the pH of the hydrolysis vessel 3 or the methanogenesis vessel 2. The user may then manually clean the pH sensor of the respective vessel and place the sensor in a solution of known pH. Once the pH of the solution has been calculated, the user may record the sensor pH in the pH calibration panel 400. When the sensor volts have stabilized, the user may engage the write table button found on the pH calibration panel 400. For each pH that the pH calibration panel 400 records, a corresponding amount of volts may be recorded as well. The system 100 may then establish a scale and offset using the values in the tables; to save the scale and offset, a user may engage the save calibration icon. Once saved, the scale and offset may be displayed adjacent the calibration table. A user may also reset the calibration by engaging the reset table icon which clears the table of numeric results.

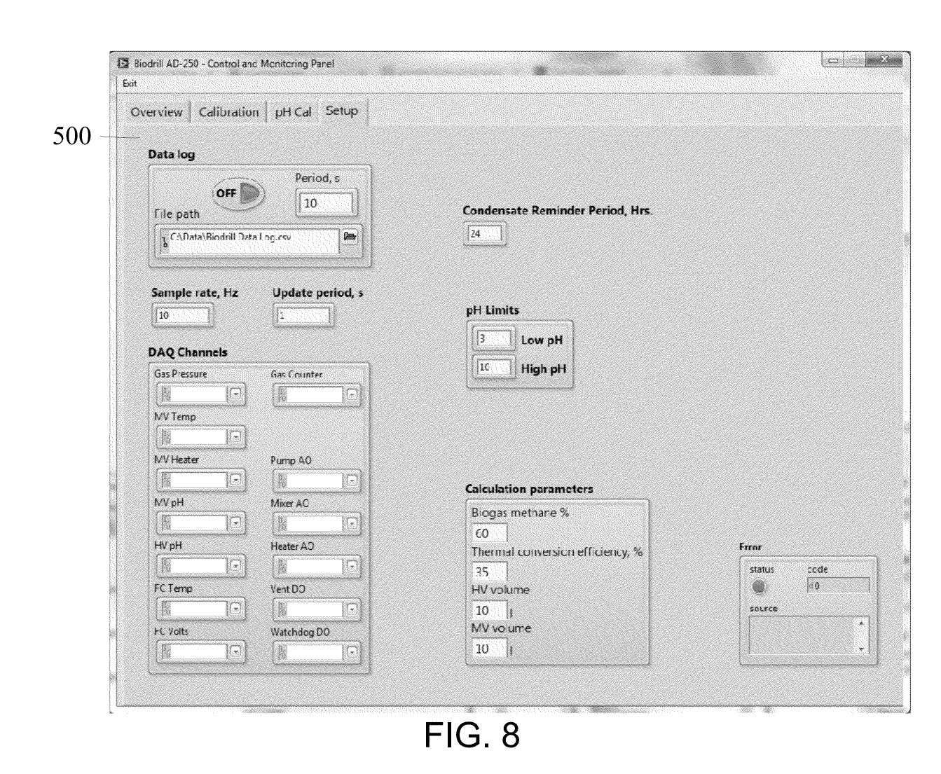

FIG. 8 depicts a screen shot displayed on the monitor 148 of the computing system showing a setup panel 500 in accordance with embodiments. The data log section may allow a user to control whether the system may log data. A user may set the amount of time that the system will log information (period) as well as set a file path for the information. In the screen shot of FIG. 8, the file may be saved in the system memory 108 of the system 100 (C drive) under the data subfolder and may be saved as a .csv file. Sample rates, period updates, a condensate reminder period, and pH limits may be manually entered by a user. The user may further enter calculation parameters such as biogas methane percent, thermal conversion efficiency percent, hydrolysis vessel volume, and methanogenesis volume. The DAQ channels section may display various parameters relative to the system such as gas pressure, methanogenesis vessel temperature, methanogenesis vessel heater, methanogenesis vessel pH, hydrolysis pH, FC temperature, FC volts, gas counter, pump AO, mixer AO, heater AO, vent DO, and watchdog DO.

In embodiments, the system 100 may comprise other panels that may be utilized by a user in order to control the system 180, manipulate the system 180, etc.

For the purposes of this disclosure, the term “TS-AD250” may be the anaerobic digester system setup in connection with the computing system 100.

For the purposes of this disclosure, the terms “anaerobic digester system”, “anaerobic digester”, and “anaerobic digester/bioreactor” may be synonymous.

For the purposes of this disclosure, the terms “MV” and “methanogenesis vessel” may be synonymous.

For the purposes of this disclosure, the terms “HV” and “hydrolysis vessel” may be synonymous.

1. A bioreactor system, as substantially shown and described.

2. A method, as substantially shown and described.

3. Computer readable code, as substantially shown and described.

4. The system of claim 1, further comprising:
a computing system;
and
a bioreactor in operable communication with the computing system, wherein the computing system is operable for controlling the bioreactor system.

5. The method of claim 2, further comprising:
providing a bioreactor;
and
providing an operating system in operable communication with the bioreactor, the operating system in communication with the bioreactor.
